(19) United States
(12) Plant Patent Application Publication
(10) Pub. No.: US 2008/0127385 P1
(43) Pub. Date: May 29, 2008

(54) Pelargonium plant named 'Fiscody'

(76) Inventor: Angelika Utecht, Montabaur (DE)

Correspondence Address: JONDLE & ASSOCIATES P.C., 858 HAPPY CANYON ROAD SUITE 230, CASTLE ROCK, CO, 80108, US

(21) Appl. No.: 11/528,939

(22) Filed: Sep. 28, 2006

(51) Int. Cl. A01H 5/00

(52) U.S. Cl. PLT/329

(57) ABSTRACT

A new cultivar of pelargonium particularly characterized by having brilliant, deep-purple, semi-double flowers, floriferous with small, compact umbles, medium-green foliage with weak zonation, a medium sized, well-branched, spreading to trailing plant habit and a mid-season flowering response, is disclosed.

### GENUS AND SPECIES

Pelargonium peltatum

### VARIETY DENOMINATION

‘Fiscody’

### BACKGROUND OF THE NEW PLANT

The present invention comprises a new and distinct cultivar of pelargonium, botanically known as Pelargonium peltatum, and hereinafter referred to by the cultivar name ‘Fiscody’. The new cultivar is a product of a planned breeding program which had the objective of creating new varieties having bluish-purple flowers and fairly early flowering response. ‘Fiscody’ was discovered as a seedling resulting from the cross of the female parent ‘195-421-14’ (unpatented), a proprietary pelargonium plant having pink. single-type flowers, and the male parent ‘Kleblue’ (unpatented), a proprietary pelargonium plant having bluish-purple, double flowers.

The new cultivar was created in 2002 in Hillscheid, Germany and has been asexually reproduced repeatedly by vegetative cuttings in Galdar, Gran Canaria, Spain, and Hillscheid, Germany over a three-year period. It has been found to retain its distinctive characteristics through successive asexual propagations. ‘Fiscody’ reproduces true to type in successive generations of asexual reproduction.

Plant Breeder's Rights for this cultivar were applied for in Germany on Apr. 4, 2005 and Canada on Apr. 20, 2005.

### SUMMARY OF THE INVENTION

The following are the most outstanding and distinguishing characteristics of this new cultivar when grown under normal horticultural practices in Hillscheid, Germany.

1. Brilliant, deep-purple, semi-double flowers;

2. Floriferous with small, compact umbels;

3. Medium-green foliage with weak zonation;

4. A medium-sized, well-branched, spreading to trailing plant habit; and

5. A mid-season flowering response.

### DESCRIPTION OF THE PHOTOGRAPHS

This new pelargonium plant is illustrated by the accompanying photographs which show overall plant habit including inflorescences, buds, and foliage of the plant; the colors shown are as true as can be reasonably obtained by conventional photographic procedures. The photographs are of three, 25-week-old, pinched plants grown from rooted cuttings, in a hanging basket taken in late summer 2005 in Hillscheid, Germany and grown under greenhouse and outdoor conditions which approximate those generally used in commercial practice.

FIG. 1 shows the overall plant habit with blooms, buds and foliage

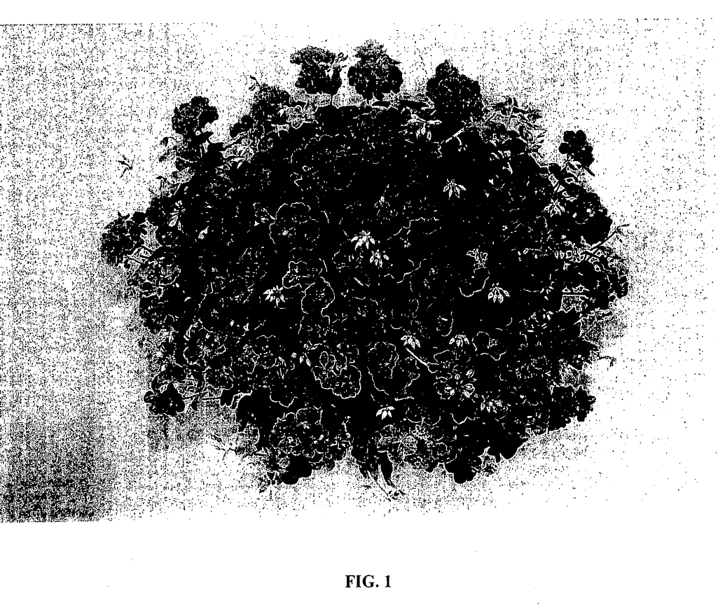

FIG. 2 shows a close-up view of an inflorescence

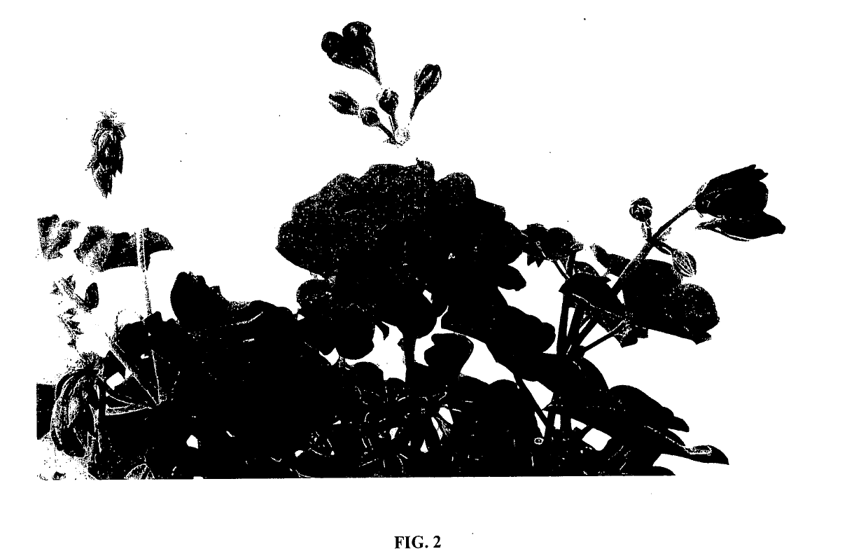

### DESCRIPTION OF THE NEW CULTIVAR

The following detailed description sets forth the distinctive characteristics of ‘Fiscody’. The data which define these characteristics were collected from asexual reproductions carried out in Hillscheid, Germany. The plant history was taken on 15-week-old, un-pinched plants in 12-cm pots in a greenhouse during late May 2005. Color readings were taken under natural light in late May from flowers grown in the greenhouse. Color references are primarily to the RHS Colour Chart of The Royal Horticultural Society of London (R.H.S.) (2001).

### DETAILED BOTANICAL DESCRIPTION +

Classification:
   Family.—Geraniaceae.
   Botanical.—Pelargonium peltatum L'Héritier.
   Common name.—Ivy geranium.
Parentage:
   Female parent.—‘195-421-14’ a proprietary pelargonium plant having pink, single-type flowers (unpatented).
   Male parent.—‘Kleblue’, a proprietary pelargonium plant having pale-bluish-purple, double flowers (unpatented).
Plant:
   Form.—Shrub, self-branching, decumbent and trailing
   Branching habit.—9 to 12 branches per plant.
   Height (measured from base of the stem to the tips of the branches, excluding the inflorescence).—14.3 cm.
   Width (plant diameter).—31.7 cm.
   Time to produce a flowering plant.—Plants had an average of 0.5 opened inflorescences at about 12 weeks after planting rooted cuttings in Hillscheid, Germany.
   Outdoor plant performance.—Plants continuously flower; a count in late May of 2005 in Hillscheid, Germany produced about 3 to 5 inflorescences per plant; in late summer, plants about 30-weeks old, produced about 15 to 20 inflorescences per plant.
Leaves:
   Arrangement.—Alternate, opposite at flowering branches.
   Immature leaf color.—Grass-green. Upper surface.—RHS 143A Lower surface.—RHS 143B.
   Mature leaf color.— Upper surface: RHS 137D (medium-green) Lower surface: RHS 143A.
   Zonation color.—RHS 166A (brown) with medium distinctness.
   Zonation diameter.— Inner diameter of ring: 2.0 cm to 2.5 cm Band or belt of zonation: 1.0 cm.
   Size.— Length: 5.5 cm Width: 8.9 cm.
   Shape.—Ivy-shaped, with distinct lobes and with a gap between lowest lobes.
   Apex.—Rounded, 5-lobed.
   Base.—Cordate.
   Margin: Entire
   Texture: Upper surface is leathery, smooth, and slightly glossy
   Petioles: Color: RHS 143B (light-green) Length: 4.0 cm to 6.0 cm Diameter: 0.25 cm Texture: Very short pubescence
Stems:
   Stem color.—RHS 143B (green).
   Length.—22.0 cm to 25.0 cm.
   Diameter.—0.4 to 0.5 cm at midpoint.
   Internode length.—0.5 cm to 1.0 cm.
   Texture.—Appear smooth, young stems with slight pubescence.
Inflorescence bud: (just before petals unfold)
   Shape.—Elliptical.
   Size.— Length: 1.5 cm Width: 0.8 cm.
   Color of sepals.—RHS 143C (light-green).
   Color of petals.—RHS N74A.
Inflorescence:
   Inflorescence type.—An umbel composed of 5 to 9 flowers.
   Umbel.— Shape: Nearly semi-spherical, tight, relatively small Diameter: 7.8 cm Height (depth): 4.8 cm
   Lastingness of umbel on the plant.—About 14 to 16 days
   Peduncle.— Color: RHS 143C (light-green) Length: 12.7 cm Diameter: 0.30 cm Texture: With weak pubescence.
   Pedicel.— Color: RHS 143B (grass-green) Anthocyanin: Absent Length: 2.5 cm Diameter: 0.15 cm Texture: Dense pubescence.
Corolla:
   Shape of corolla.—Wide open, often with a gap between upper petals and lower petals.
   Form.—Semi-double to double.
   Diameter.—5.0 cm.
   Depth.—1.2 to 1.5 cm.
   Number of petals.—10 to 13.
   Petaloids.— Number: 0 to 2. Shape: Variable, shorter and narrower than the petals Color: RHS N74B.
   Lastingness of individual flowers on plant.—8 days at 18° C.
   Fragrance.—None.
Petals:
   Shape.— Upper petals: Spathulate Lower petals: Obovate.
   Apex.—Rounded
   Base.—Acute or Acuminate
   Margin.—Entire
   Texture.—Smooth, slightly glossy
   Upper petals.— Length: 2.7 cm to 2.8 cm Width: 1.7 cm to 1.8 cm Color: Upper surface: RHS N74A (intense purple) Lower surface: RHS N74B (light purple), deep pink veins, RHS 67A towards the base Markings: A small dark dot, RHS 59A, in the middle of the petal and a pair of fine, dark-veins, RHS 53A, from the base to the middle.
   Lower petals.— Length: 2.3 cm to 2.4 cm Width: 1.7 cm to 1.9 cm Color: Upper surface: RHS N74A Lower surface: RHS N74B Markings: No distinct markings.
Sepals:
   Number.—5.
   Color (both surfaces).—RHS 143A to RHS 143B (grass-green).
   Anthocyanin.—Absent.
   Length.—1.0 cm to 1.2 cm.
   Width.— Largest upper sepal: 0.5 cm Other sepals: 0.3 cm.
   Shape.—Lanceolate.
   Apex.—Acute.
   Base.—Fused.
   Margin.—Entire.
   Texture.—Short pubescence and a few longer hairs.
Reproductive organs:
   Androecium.— Number of anthers: 5 to 7 Filament length: 0.6 to 0.8 cm Filament color: Mainly RHS 155D (white) to RHS 73A (purple-pink) at upper end Pollen color: RHS 26A (yellow-orange) Pollen amount: Moderate
   Gynoecium.— Pistil: Number: 1 Length: 1.0 cm Stigma color: RHS 71 A (deep-purple) Stigma shape: 5 to 6 lobed Style color: RHS 73D (pale-pink) Style length: 0.4 cm Style shape: Filiform (filament-like) with the lobes of the stigma upright or at right angles.
Fruit and seed set: No seed set observed.
Disease and insect resistance: No particular resistance or susceptibility has been observed.

### COMPARISON WITH PARENTAL AND COMMERCIAL CULTIVARS

‘Fiscody’ differs from the female parent ‘195-421-14’ (unpatented) by having deep-purple, semi-double to double flowers, while ‘195-421-14’ has pink, single-type flowers.
‘Fiscody’ differs from the male parent ‘Kleblue’ (unpatented) by having a slightly deeper flower color, flowers with fewer petals, stronger markings on upper petals and only 5 sepals compared to ‘Kleblue’.
‘Fiscody’ differs from the commercial variety ° ‘Fiscomedy’ (U.S. Plant Pat. No. 9,394) by having fewer petals, smaller but more numerous inflorescences and an earlier flowering response than ‘Fiscomedy’. In addition, ‘Fiscody’ has more upright directed branches and inflorescences, while ° Fiscomedy' has more creeping and trailing branches and inflorescences.

I claim:

1. A new and distinct cultivar of pelargonium plant as shown and described herein.
